(19) United States
(12) Patent Application Publication
(10) Pub. No.: US 2022/0031879 A1
(43) Pub. Date: Feb. 3, 2022

(54) STERILIZING LAMP

(71) Applicants: Quark Technology Co., Ltd, Okayama (JP); Tazmo Co., Ltd, Okayama (JP)

(72) Inventor: Masaru NAKAMURA, Okayama (JP)

(21) Appl. No.: 17/035,861

(22) Filed: Sep. 29, 2020

(30) Foreign Application Priority Data
Aug. 3, 2020 (JP) 2020-131749

(51) Int. Cl. A61L 2/10

(52) U.S. Cl. CPC A61L 2/10; A61L 2202/121; A61L 2202/11

(57) ABSTRACT

A sterilizing lamp includes a discharge tube, a ground electrode part, a high-voltage electrode part, a safety cover, and a light shield. The discharge tube is filled with discharge gas for generating excimer light. The ground electrode part and the high-voltage electrode part generate discharge in the discharge tube to excite the discharge gas. The safety cover is made of an electrically insulating organic material and covers the high-voltage electrode part. The light shield is made of an electrically insulating inorganic material and intervenes between the discharge tube and the safety cover to block the excimer light traveling from the discharge tube toward the safety cover.

### BACKGROUND OF THE INVENTION

### 1. Field of the Invention

The present invention relates to a sterilizing lamp that supplies ultraviolet light for sterilization.

### 2. Description of Related Art

Ultraviolet light shows high sterilizing effect against pathogens such as germs or viruses, so that it is used for disinfection of sanitary fixtures, water, air, and others in many cases. However, as ultraviolet light has conventionally been considered to be harmful to human bodies (mainly skins, for example), use of ultraviolet light for disinfection has been limited to subjects (sanitary fixtures, water, air, and others) other than human bodies.

Ultraviolet light is used for various types of process in fields such as semiconductor manufacturing technology and environmental technology. In the field of semiconductor manufacturing technology, ultraviolet light is used for process such as dry cleaning, surface activation process, and soft ashing. In the field of environmental technology, ultraviolet light is used for process such as ozone generation, decontamination of water and atmosphere, and ultrapure water production. Ultraviolet light to be used suitably for such purposes is high-energy ultraviolet light of a short wavelength (vacuum ultraviolet light, for example). The present inventor is developing an excimer light irradiation device that generates excimer light using discharge gas to be applied as a UV irradiation device used in fields such as semiconductor manufacturing technology and environmental technology described above (see Japanese Patent Laid-Open Publication No. 2020-68133, for example).

Recent research has revealed that, as long as ultraviolet light is in a wavelength range up to 230 nm, this ultraviolet light is harmless to human bodies. This shows that ultraviolet light in such a wavelength range is available for use in disinfection of hands and others. The wavelength range up to 230 nm is a range in which the excimer light irradiation device developed by the present inventor is used favorably.

In this regard, the present inventor is thinking of applying the excimer light irradiation device developed by the present inventor to a sterilizing lamp (sterilizing lamp for consumer use, for example) available for use in disinfection of hands and others. However, the present inventor has found that size reduction, cost reduction, and easiness of manufacture are required to be fulfilled for such a sterilizing lamp, and that various attempts differing from those for industrial use are required to be made for the fulfillment.

### SUMMARY OF THE INVENTION

A sterilizing lamp according to the present invention includes a discharge tube, a ground electrode part, a high-voltage electrode part, a safety cover, and a light shield. The discharge tube is filled with discharge gas for generating excimer light. The ground electrode part and the high-voltage electrode part generate discharge in the discharge tube to excite the discharge gas. The safety cover is made of an electrically insulating organic material and covers the high-voltage electrode part. The light shield is made of an electrically insulating inorganic material and intervenes between the discharge tube and the safety cover to block the excimer light traveling from the discharge tube toward the safety cover.

### BRIEF DESCRIPTION OF THE DRAWINGS

FIG. 1A is a vertical sectional view of a sterilizing lamp according to an embodiment, and FIG. 1B is a front view of the sterilizing lamp.

FIG. 2 is a vertical sectional view of a sterilizing lamp according to a first modification.

FIG. 3 is a vertical sectional view of a sterilizing lamp according to a second modification.

FIGS. 4A and 4B are vertical sectional views showing two examples of a sterilizing lamp according to a fifth modification.

### DETAILED DESCRIPTION OF THE EMBODIMENTS

### [1] Embodiment

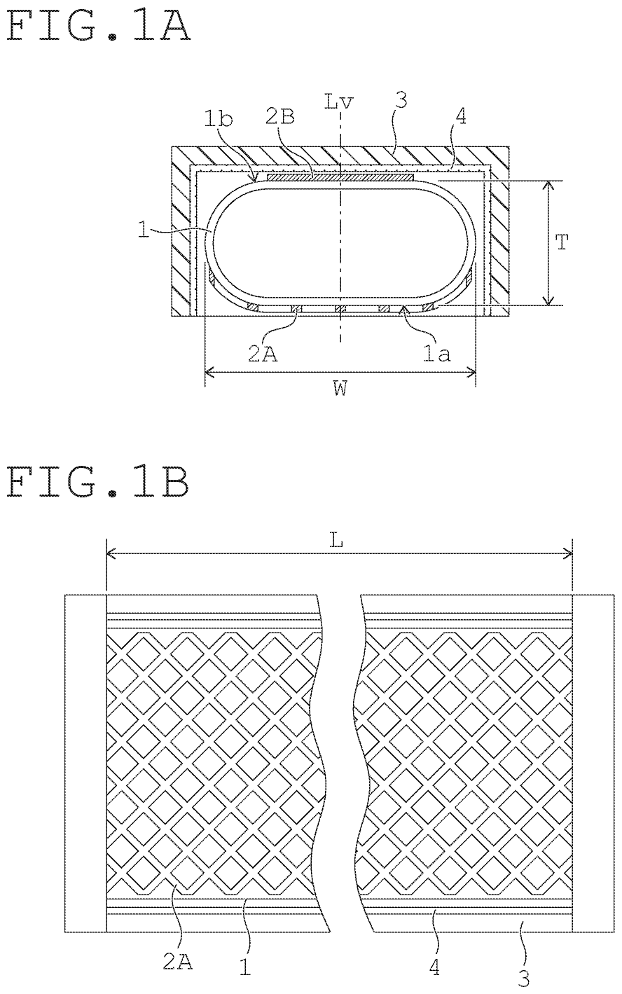

FIG. 1A is a vertical sectional view of a sterilizing lamp according to an embodiment. FIG. 1B is a front view of the sterilizing lamp. As shown in these drawings, the sterilizing lamp of the embodiment includes a discharge tube 1, a ground electrode part 2A, a high-voltage electrode part 2B, a safety cover 3, and a light shield 4.

<Discharge Tube>

The discharge tube 1 is a tube container made of quartz or synthetic quartz having excellent permeability to ultraviolet light. The discharge tube 1 used in the embodiment is a single-lumen tube having a track cross-sectional shape. Regarding the size of the discharge tube 1, a width W of the cross section (see FIG. 1A) is from 10 to 100 mm, a height T of the cross section (see FIG. 1A) is from 2 to 10 mm, and a length L in a lengthwise direction (see FIG. 1B) is from 20 to 1000 mm. This size is smaller than the size of a discharge tube used in an excimer light irradiation device for industrial use. A material for forming the discharge tube 1 is not limited to quartz or synthetic quartz but it may be changed to a different material, if appropriate, such as fluoride that may be calcium fluoride, magnesium fluoride, or lithium fluoride, for example. The cross-sectional shape of the discharge tube 1 is not limited to a track shape but it may be changed to a different shape, if appropriate, such as a perfect circular shape, an oval shape, a flat shape, or a rectangular shape, for example.

The discharge tube 1 is filled with discharge gas for generating excimer light. As an example, the discharge tube 1 is filled with discharge gas by sealing the discharge gas in the discharge tube 1. As another example, the discharge tube 1 may always be filled with new discharge gas by introducing and ejecting discharge gas into and out of the discharge tube 1.

In the discharge tube 1, excimer light having a wavelength responsive to the type of discharge gas filling the discharge tube 1 is generated by discharge described later. Thus, selecting the type of discharge gas in response to a purpose of use makes it possible to generate excimer light in the discharge tube 1 having a wavelength suitable for the purpose of use. In the embodiment, to make excimer light available for use in disinfection of hands and others, gas such as krypton chloride (KrCl), xenon (Xe), or krypton bromide (KrBr) gas is used as discharge gas to generate excimer light in a wavelength range (up to 230 nm) harmless to human bodies. As an example, excimer light having a wavelength of 172 nm or 222 nm is generated in the discharge tube 1. In particular, excimer light having a wavelength of 172 nm can be used for generating ozone.

<Ground Electrode Part and High-Voltage Electrode Part>

The outer peripheral surface of the discharge tube 1 has two surfaces (in the embodiment, two planar surfaces corresponding to linear sections of the track forming the cross-sectional shape) intersecting a virtual line Lv (see FIG. 1A) extending in a height direction passing through the center of the discharge tube 1. In this embodiment, these two surfaces are called a front surface 1a and a back surface 1b of the discharge tube 1, and the ground electrode part 2A is formed at the front surface 1a and the high-voltage electrode part 2B is formed at the back surface 1b. Moreover, these electrode parts are formed into layered shapes by vapor deposition of electrode materials on the front surface 1a and the back surface 1b. Aluminum is used as the electrode materials.

In this configuration, application of a high voltage (from 1 to 5 kV, for example) to the high-voltage electrode part 2B generates discharge between the ground electrode part 2A and the high-voltage electrode part 2B. This generates discharge in the discharge tube 1 to excite the discharge gas. When the excited discharge gas is thereafter returned to its ground state, excimer light is generated.

In the embodiment, the ground electrode part 2A is formed into a mesh-like shape so as to emit much of the excimer light generated in the discharge tube 1 toward the front surface 1a of the discharge tube 1.

The electrode material is not limited to aluminum but it may be changed to a different conductive material, if appropriate, such as an aluminum alloy, copper, copper oxide, gold, silver, stainless steel, or an alloy of these materials, for example. A method of forming the electrode parts is not limited to vapor deposition described above but it may be changed, if appropriate, to a method of bonding a thin film of a conductive material to the discharge tube 1, for example. The ground electrode part 2A is not limited to a part formed into a mesh-shape but it may be changed, if appropriate, to a part allowing transmission of excimer light while functioning as an electrode that may be an electrode part with a plurality of openings formed uniformly over the entire area of the part.

<Safety Cover>

The safety cover 3 is a cover covering the high-voltage electrode part 2B to prevent an electrical shock during voltage application. The safety cover 3 is made of an electrically insulating organic material. While the electrically insulating organic material is not particularly limited, polyetheretherketone (PEEK), polyethylene (PE), polytetrafluoroethylene (PTFE), or acrylonitrile butadiene styrene (ABS) resin may be used, for example.

These organic materials fulfill the size reduction, cost reduction, and easiness of manufacture of the safety cover 3, and eventually fulfill the size reduction, cost reduction, and easiness of manufacture of the sterilizing lamp. On the other hand, these organic materials are prone to decomposition to be caused by ultraviolet light. Hence, if excimer light leaking from the discharge tube 1 toward the back surface 1b reaches the safety cover 3, the safety cover 3 is decomposed by the excimer light to be deteriorated. In particular, as the energy of ultraviolet light increases in a short wavelength range up to 230 nm, even a covalent bond having high bond energy such as a CH bond or an OH bond is still decomposed by the ultraviolet light.

To prevent such deterioration of the safety cover 3, the sterilizing lamp of the embodiment is provided with the light shield 4 described below.

<Light Shield>

The light shield 4 is a part intervening between the discharge tube 1 and the safety cover 3 to block excimer light traveling from the discharge tube 1 toward the safety cover 3. The light shield 4 is made of an electrically insulating inorganic material. The light shield 4 used in the embodiment is a light shielding plate formed into an angular U-shape extending along the inner surface of the safety cover 3. While the electrically insulating inorganic material is not particularly limited, it is preferably a material such as silicon having high reflectivity to ultraviolet light, mainly to light in a short wavelength range up to 230 nm.

The shape of the light shield 4 is not limited to an angular U-shape but it may be changed, if appropriate, to a round U-shape or a flat-plate shape in response to the shape of the discharge tube 1 or the safety cover 3. The light shield 4 may be formed into a layered shape by applying a coating made of an electrically insulating inorganic material to the inner surface of the safety cover 3 or to the outer surface of the discharge tube 1 or that of the high-voltage electrode part 2B.

In the sterilizing lamp of the embodiment, as excimer light in a wavelength range (up to 230 nm) harmless to human bodies is generated in the discharge tube 1 and emitted toward the front surface 1a of the discharge tube 1, the excimer light emitted from the sterilizing lamp becomes available for use in disinfection of hands and others.

In the sterilizing lamp described above, covering the high-voltage electrode part 2B with the safety cover 3 ensures safety of the sterilizing lamp. Further, using the organic material for forming the safety cover 3 fulfills the size reduction, cost reduction, and easiness of manufacture of the safety cover 3. This eventually fulfills the size reduction, cost reduction, and easiness of manufacture of the sterilizing lamp. Additionally, as excimer light traveling from the discharge tube 1 toward the safety cover 3 is blocked by the light shield 4, the safety cover 3 is less prone to deterioration (decomposition) to be caused by the excimer light leaking from the discharge tube 1 toward the back surface 1b. Further, using the inorganic material for forming the light shield 4 functions to reduce the occurrence of deterioration (decomposition) of the light shield 4 itself to be caused by the excimer light.

As described above, the configuration of the embodiment realizes the sterilizing lamp (sterilizing lamp for consumer use, for example) available for use in disinfection of hands and others using the excimer light irradiation device.

### [2] Modifications

### [2-1] First Modification

The sterilizing lamp described above is not limited to the configuration in which the ground electrode part 2A and the high-voltage electrode part 2B are formed integrally with the discharge tube 1 by a step such as vapor deposition but these electrode parts may be devices different from and detachable from the discharge tube 1. Moreover, the devices may be provided to the safety cover 3. A specific example will be described next.

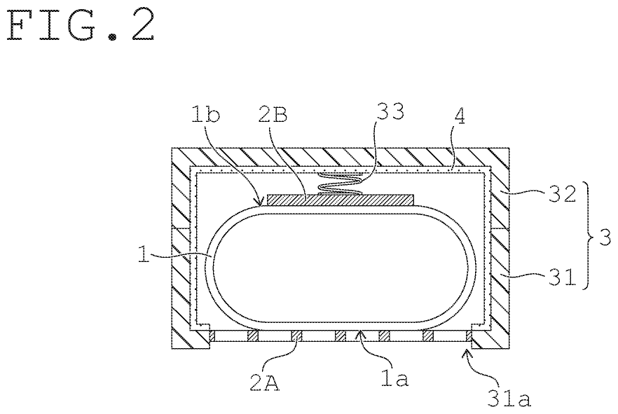

FIG. 2 is a vertical sectional view of a sterilizing lamp according to a first modification. As shown in FIG. 2, in the sterilizing lamp of the first modification, the safety cover 3 is composed of a front cover 31 covering the discharge tube 1 at the front surface 1a, and a back cover 32 covering the discharge tube 1 at the back surface 1b. The front cover 31 is provided with an opening 31a for causing excimer light emitted toward the front surface 1a of the discharge tube 1 to pass through. A mesh member having conductivity is fitted as the ground electrode part 2A in the opening 31a. A plate member having conductivity is provided as the high-voltage electrode part 2B to the inner surface of the back cover 32.

In this configuration, the ground electrode part 2A and the high-voltage electrode part 2B can be arranged at the front surface 1a of the discharge tube 1 and at the back surface 1b of the discharge tube 1 respectively by simply inserting the discharge tube 1 into the front cover 31 and then combining the front cover 31 and the back cover 32 together without the need of forming the ground electrode part 2A and the high-voltage electrode part 2B integrally with the discharge tube 1 through a step such as vapor deposition. This eliminates the step such as vapor deposition on the discharge tube 1 to facilitate manufacture of the sterilizing lamp.

In order for the ground electrode part 2A and the high-voltage electrode part 2B to tightly contact the front surface 1a and the back surface 1b of the discharge tube 1 respectively when the front cover 31 and the back cover 32 are combined together, a biasing member 33 (compression spring, for example) may be provided to the back cover 32 for biasing the high-voltage electrode part 2B toward the discharge tube 1.

In the sterilizing lamp of the first modification, one of the ground electrode part 2A and the high-voltage electrode part 2B may be formed integrally with the discharge tube 1 by vapor deposition, for example, and the other of the electrode parts may be provided to the safety cover 3 as the device different from and detachable from the discharge tube 1.

### [2-2] Second Modification

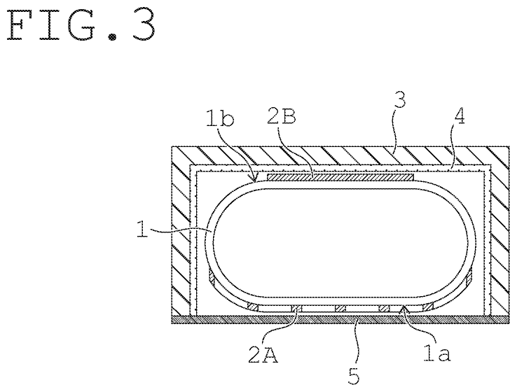

FIG. 3 is a vertical sectional view of a sterilizing lamp according to a second modification. As shown in FIG. 3, the sterilizing lamp may further include a filter 5 provided outside the discharge tube 1 (more specifically, the front surface 1a of the discharge tube 1) and functioning as removal means that removes light in a long wavelength range greater than 230 nm. In this case, excimer light generated in the discharge tube 1 and having passed through the filter 5 is supplied as ultraviolet light for sterilization from the sterilizing lamp.

This sterilizing lamp can be used for supplying excimer light in a wavelength range (up to 230 nm) assured of being harmless to human bodies as ultraviolet light for sterilization.

### [2-3] Third Modification

The filter 5 is not always configured as a device different from and detachable from the discharge tube 1 but it may be formed integrally with the discharge tube 1. More specifically, the filter 5 may be a deposited film formed by vapor deposition of hafnium, for example, on the front surface 1a of the discharge tube 1.

### [2-4] Fourth Modification

In the sterilizing lamp described above, the discharge tube 1 may be formed using a material allowing suppression of transmission of ultraviolet light having a wavelength greater than 230 nm. Such a material may be prepared by adding an element such as hafnium to quartz, synthetic quartz, or fluoride, for example. In this case, the discharge tube 1 itself functions as removal means that removes light in a long wavelength range greater than 230 nm.

In this sterilizing lamp, most of excimer light emitted from the discharge tube 1 is light in a wavelength range (up to 230 nm) harmless to human bodies. Thus, even in the absence of the filter 5, excimer light emitted from the discharge tube 1 can still be supplied as it is as ultraviolet light for sterilization.

### [2-5] Fifth Modification

In the sterilizing lamp of the foregoing embodiment, the size of the discharge tube 1 is reduced for fulfilling the size reduction of the sterilizing lamp. On the other hand, the size reduction of the discharge tube 1 reduces a distance between the ground electrode part 2A and the high-voltage electrode part 2B, which increases a probability of the occurrence of creeping discharge at the discharge tube 1. In this regard, the discharge tube 1 may be provided with prevention means that prevents creeping discharge. Two examples of the prevention means will be described next.

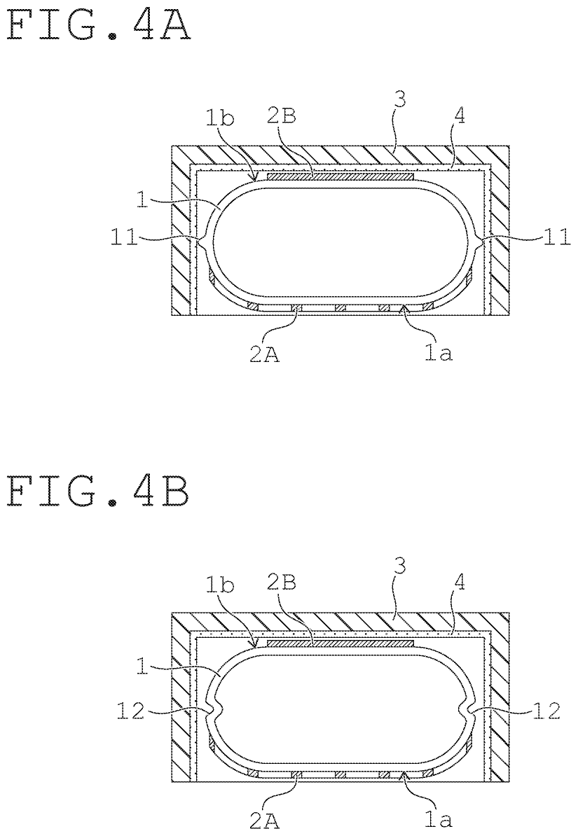

FIGS. 4A and 4B are vertical sectional views showing two examples of a sterilizing lamp according to a fifth modification.

As shown in FIG. 4A, a projection 11 for increasing an insulation distance between the ground electrode part 2A and the high-voltage electrode part 2B may be provided as an example of the prevention means to the discharge tube 1. As a specific example, the projection 11 can be formed by attaching a glass rod, for example, to a side surface of the discharge tube 1. As shown in FIG. 4B, a groove 12 for increasing an insulation distance between the ground electrode part 2A and the high-voltage electrode part 2B may be provided as another example of the prevention means to the discharge tube 1. The prevention means is not limited to these examples but it can be changed, if appropriate, in response to the shape of the discharge tube 1, for example, so as to increase an insulation distance between the ground electrode part 2A and the high-voltage electrode part 2B.

### [2-6] Sixth Modification

In the sterilizing lamp described above, the discharge tube 1 is not limited to a tube-like shape but it may be changed, if appropriate, to a flat type having a shape like a thin box.

### [2-7] Other Modifications

In the sterilizing lamp described above, the safety cover 3 itself may be made of an electrically insulating inorganic material. In this case, even if excimer light reaches the safety cover 3, the safety cover 3 is still less prone to deterioration (decomposition) to be caused by the excimer light. This eliminates the need for the light shield 4.

The foregoing descriptions of the embodiment and modifications are illustrative in all points and should not be construed as being restrictive. The scope of the present invention is defined not by the foregoing embodiment or modifications but by the claims. Further, the scope of the present invention is intended to include all modifications within the scope of the claims and within the meanings and scopes of equivalents.

The foregoing descriptions of the embodiment and modifications include not only the invention described in the claims but also include a configuration as an invention made by extracting the removal means or prevention means described above as an alternative feature to the light shield 4.

What is claimed is:

1. A sterilizing lamp comprising:
a discharge tube filled with discharge gas for generating excimer light;
a ground electrode part and a high-voltage electrode part that generate discharge in the discharge tube to excite the discharge gas;
a safety cover made of an electrically insulating organic material and covering the high-voltage electrode part; and
a light shield made of an electrically insulating inorganic material and intervening between the discharge tube and the safety cover to block the excimer light traveling from the discharge tube toward the safety cover.

2. The sterilizing lamp according to claim 1, wherein the inorganic material has high reflectivity to light in a short wavelength range up to 230 nm.

3. The sterilizing lamp according to claim 1, further comprising:
removal means that removes light in a long wavelength range greater than 230 nm, wherein
excimer light generated in the discharge tube and having passed through the removal means is supplied as ultraviolet light for sterilization.

4. The sterilizing lamp according to claim 2, further comprising:
removal means that removes light in a long wavelength range greater than 230 nm, wherein
excimer light generated in the discharge tube and having passed through the removal means is supplied as ultraviolet light for sterilization.

5. The sterilizing lamp according to claim 1, wherein
the discharge tube is provided with prevention means that prevents creeping discharge.
